(19) United States
(12) Patent Application Publication
(10) Pub. No.: US 2012/0148677 A1
(43) Pub. Date: Jun. 14, 2012

(54) CONTROLLED RELEASE PARTICLES CONTAINING ACID FIBROBLAST GROWTH FACTOR

(75) Inventors: Henrich Cheng, Pei-Tou Dist. (TW); Son-Haur Hsu, Taipei City (TW)

(73) Assignee: Taipei Veterans General Hospital, Taipei City (TW)

(21) Appl. No.: 13/070,080

(22) Filed: Mar. 23, 2011

Related U.S. Application Data
(60) Provisional application No. 61/421,818, filed on Dec. 10, 2010.

(51) Int. Cl. A61K 9/50; A61P 25/00; A61K 38/18

(52) U.S. Cl. 424/490; 424/491; 424/492; 424/493; 514/9.1

(57) ABSTRACT

Disclosed herein is a controlled release particle comprising a therapeutically effective amount of acid fibroblast growth factor (aFGF), entrapped by a particle composed by a biocompatible anionic biopolymer capable of binding to aFGF, and a cationic polymer. The method for manufacturing the controlled release particle and the method of using the particle for treating nervous injury are also provided.

### BACKGROUND OF THE INVENTION

Acid fibroblast growth factor (aFGF; also known as FGF-1) is a member of the FGF family that acts on a variety of cells either by stimulating proliferation or inducing differentiation, which indicates the potential of post-injured repairing properties in medical applications. It was reported in US 2004-0267289 A1 published in Dec. 20, 2004 (U.S. patent application Ser. No. 10/766,530 filed Jan. 29, 2004) that aFGF is effective in nerve root repair. However, the therapeutic use of aFGF is hindered by its short in vivo half-life due to rapid degradation after administrated to a subject.

### BRIEF SUMMARY OF THE INVENTION

This invention provides an approach to control the release of aFGF, thereby prolonging its presence in vivo after delivery.

The purpose of the present invention is to provide a controlled release particle comprising aFGF for nerve repair.

Accordingly, one aspect of the present disclosure relates to a controlled release particle comprising a therapeutically effective amount of acid fibroblast growth factor (aFGF), entrapped by a particle composed by a biocompatible anionic biopolymer capable of binding to aFGF, and a cationic polymer. This controlled release particle is useful in treating a nervous injury or in manufacturing a medicament useful in nervous injury treatment.

Another aspect of the present disclosure is to provide a method for manufacturing the controlled release particle of the invention comprising: mixing a biocompatible anionic biopolymer capable of binding to aFGF, and a cationic polymer to form a particle, and then with aFGF to allow aFGF to be entrapped by the particle.

Yet another aspect of this disclosure is to provide a method for treating a nervous injury in a subject comprising locally administrating to the nervous injury with the controlled release particle as disclosed herein.

It is to be understood that both the foregoing general description and the following detailed description are exemplary and explanatory only and are not restrictive of the invention.

### BRIEF DESCRIPTION OF THE SEVERAL VIEWS OF THE DRAWINGS

The foregoing summary, as well as the following detailed description of the invention, will be better understood when read in conjunction with the appended drawings.

In the drawings:

FIG. 1 is an illustration of the particles according to the invention; which comprises aFGF entrapped by chitosan/heparin particles providing affinity between heparin and aFGF, as well as heparin and chitosan; wherein aFGF was entrapped by heparin/chitosan particles;

FIG. 2A provides the results of a sandwich ELISA, measuring the amount of aFGF in the particles according to the invention;

FIG. 2B shows the results of Western blot, wherein the first three bands showed the amount of aFGF attracted between heparin and chitosan in the particles of the invention, and the last three bands showed the amount of the aFGF suspended in the supernatant;

FIG. 3 are photographs showing an analysis by Western blot on the proteolytic sensitivity of intact aFGF (A) and aFGF entrapped by chitosan/heparin particles (B);

FIG. 4 is a photograph showing an analysis by Western blot on the release profile of aFGF from the controlled release particles of the invention in 1×PBS at room temperature for 20 days; wherein the pattern of Western blot indicated the amounts of aFGF contained in the particles (A) and released into the supernatant (B), respectively, and standard aFGFs were included as internal controls at the right side;

FIG. 5 provides a diagram showing that the viability of PC-12 cells treated by 6-OHDA, and then treated with free aFGF, aFGF in the particles according to the invention, and the empty particles (without aFGF), wherein one unit of aFGF contains 10 pg aFGF, and one unit of the particles contains 10 ng/ml;

FIG. 6 is the results of the CGRP staining showing the distribution of sensory axons on the cross section of spinal cord treated with unilateral rhizotomy only (A, B), empty particles (without aFGF) (C) and aFGF entrapped by chitosan/heparin particles (the particles of the invention) (D).

### DETAILED DESCRIPTION OF THE INVENTION

As used herein, the article “a” or “an” means one or more than one (that is, at least one) of the grammatical object of the article, unless otherwise made clear in the specific use of the article in only a singular sense.

The term “acid fibroblast growth factor” or “aFGF” as used herein refers to a native acid fibroblast growth factor (aFGF) or any modified peptide from the native aFGF. Particularly, the aFGF is human aFGF. The modified peptide may be obtained such as by one or more deletions, insertions or substitutions or combination thereof in the native human aFGF. In one example of the invention, the modified human aFGF is a peptide comprising a native human aFGF shortened by a deletion of 20 amino acids from N-terminal of the native human aFGF, and an addition of Alanine before the shortened native aFGF, which is described in U.S. patent application Ser. No. 12/482,041, and hereby incorporated by reference herein in its entirety.

The term “therapeutically effective amount” as used herein refers to an amount that is used for repairing neural injury, and recovering neural function in a subject in need thereof. For those skilled in the art, the therapeutically effective amount, as well as dosage and frequency of administration, may easily be determined according to their knowledge and standard methodology of merely routine experimentation.

The invention relates to a controlled release particle comprising a therapeutically effective amount of acid fibroblast growth factor (aFGF), entrapped by a particle composed by a biocompatible anionic biopolymer capable of binding to aFGF, and a cationic polymer.

According to the invention, the biocompatible anionic biopolymer capable of binding to aFGF may be any protein binding to aFGF, including but not limited to collagen, gelatin, alginate, heparin, or hyaluronan. In one embodiment of the present invention, the biocompatible anionic biopolymer is heparin. According to the invention, heparin acts as a crucial part of the particles, since it not only has negative charge to attract cationic polymer but also is capable of binding to aFGF.

The term “cationic polymer” used herein refers to a polymer carrying positive charges. In one embodiment of the present invention, the cationic polymer is chitosan. Because there is interaction between heparin and aFGF, and between heparin and chitosan, aFGF can be entrapped with chitosan and heparin, which form chitosan/heparin particles with heparin-aFGF specific affinity.

The present invention also provides a method for manufacturing the controlled release particle of the invention comprising: mixing a biocompatible anionic biopolymer capable of binding to aFGF, and a cationic polymer to form a particle, and then with aFGF to allow aFGF to be entrapped by the particle.

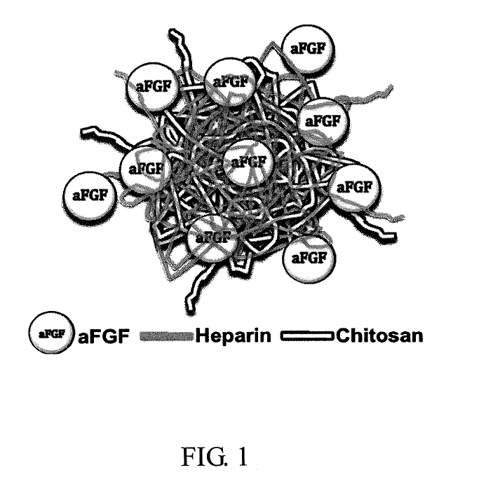

In one embodiment of the present invention, the solution containing chitosan and the solution containing heparin are mixed to form particles, and then mixed with aFGF to obtain the controlled release particle according to the invention. An illustration of the particles according to the invention is given in FIG. 1.

In one example of the invention, the activity of the chitosan/heparin/aFGF particles was tested and the particles provided a controlled release of aFGF from the particle of the present invention so as to prevent aFGF from proteolysis. Moreover, it was also proved that the particle of the invention had good bioactivity of aFGF in neutralizing the neurotoxicity of 6-hydrodopamine in PC-12. The unexpectedly good results were found in the rhizotomized rat model on the function of the controlled release particles of the invention. It was demonstrated in the invention that the controlled release particles of the invention exhibited the anti-adhesion effect which prevented damaged-tissue adhesion and decreased fibrosis, hence kept the tissue structure intact.

Accordingly, the invention also provides a method for treating a nervous injury in a subject comprising locally administrating to the nervous injury with the controlled release particle according to the invention.

The present invention is more specifically explained by the following example. However, it should be noted that the present invention is not limited to these examples in any manner.

### Example 1

### Preparation of Chitosan-Heparin Particle Preparation

Chitosan was purchased from Sigma Chemical Co. (USA). The average molecular weight (MW) was about 645,000 with the deacetylation rate greater than 85%. Chitosan was dissolved in 2% acetic acid, and applied with H₂O₂. The depolymerizing effect of H₂O₂ produced a series of low MW chitosan. The MW can be evaluated by Mark-Houwink equation with the intrinsic viscosity of the chitosan samples. After depolymerization, the chitosan was precipitated by adding NaOH solution. The precipitants were collected by centrifugation, neutralized by double-distilled water (DDW) and reserved by lyophilization. Heparin was supplied by Sigma (H3149). Before chitosan/heparin microspheres fabrication, chitosan of different MW and two kinds of heparin solution were prepared for using, separately. Heparin was directly dissolved in DDW and then dropped into chitosan solution consisting 2% chitosan and 2% acetic acid to become oppositely charged ion polymers, to form chitosan/heparin particles.

The average size of the chitosan/heparin particles as obtained was about 240 nm. The chitosan was depolymerized with H₂O₂ to obtain low MW of 75K, and then mixed with heparin solution at the ratio of 5 ml chitosan (2 mg/ml) to 2 ml heparin (1 mg/ml). The particle size does not vary from pH 5 to pH 6.5.

The chitosan/heparin particles as obtained at different concentrations were soak in 100 ng/ml aFGF solution overnight at 4° C. Then the supernatant was collected and analyzed with ELISA kit to measure the concentration of the surplus aFGF (which were not entrapped by the particles). As a result of the test, the most efficient ratio of the particles to aFGF (w/w) was 10:1.

The specific affinity of the particles of the invention was also tested by western blot assay. The samples were separated on 12% SDS-page and then transferred to nitrocellulose membrane (Millipore). The membrane was then incubated in blocking buffer (0.1 M PBS, 0.1% Tween 20 and 5% milk power) for 1 hour at room temperature. After blocking, primary antibody (R&D, AF232) were diluted at 1:1000 in blocking buffer and incubated for 2 hours, followed by three washes in PBST (0.1 M PBS with 0.1% Tween 20) for 10 min. And then the membrane was incubated in HRP-conjugated secondary antibody (1:2000 from Jackson 705-035-003) for 2 hours. After washing four times for 10 min. in PBST, enhanced chemiluminescence (ECL) was used for antigen detection.

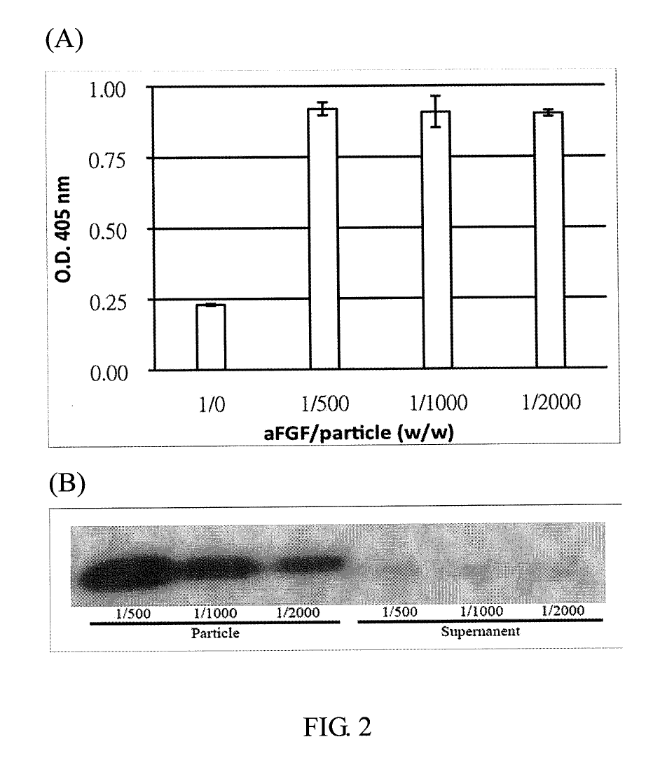

As shown in FIG. 2, most of the aFGF were entrapped by the Chitosan-Heparin particles disclosed in Example 1 above.

### Example 2

### Stabilization of aFGF in the Chitosan-Heparin Particles

In order to test how well of the particles can protect aFGF from an enzyme digestion, we mixed aFGF or the particles entrapping aFGF with a protein enzyme, trypsin. The aFGF and trypsin (Sigma, T1426) was mixed with the ratio of 1:400 in PBS bathed at 37° C. Ten microliters of product was taken out at various time intervals to mix with 10 ul of 2×SDS-sample buffer. The solution was then immediately being boiled for 5 mins to cease enzyme reaction.

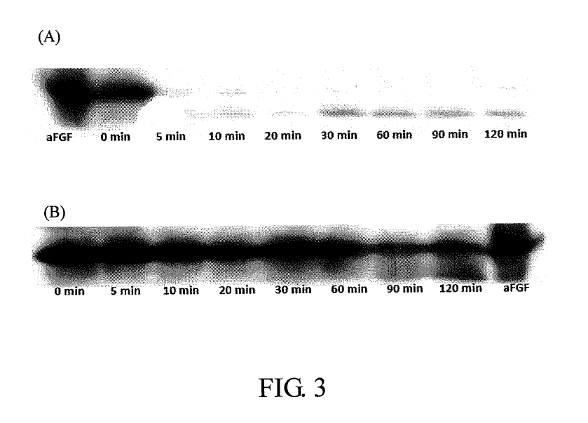

As shown in the results, the binding of aFGF to heparin increased the stability to overcome the challenge in vivo. To simulate physiological environment, the experiment was held at 37° C. The intact aFGF and aFGF in the particles of the invention were digested by Trypsin respectively, and aFGF antibody was used to detect the remaining aFGF. With the increase in time of enzyme digestion, the amount of aFGF decreases. As shown in FIG. 3A, the intact aFGF (approximately 16 kD) almost disappeared after 5 min of digestion, whereas the band of decomposed aFGF slightly darkens as indicated by the arrow head. In FIG. 3B, where the aFGF was protected by chitosan/heparin particle, the aFGF remains clearly visible after two hours of digestion. It was indicated that the particles of the invention provided significant protection to aFGF.

### Example 3

### Prolonged Release of aFGF

To check the release of aFGF from the particles of the invention, the particles of the invention in PBS was divided into 10 vials and placed at room temperature. One of the 10 vials was centrifuged every other day, and the supernatant and palate were separated in different container and preserved in −20° C. After 20 days, the 10 sets of samples were analyzed with aFGF western blot.

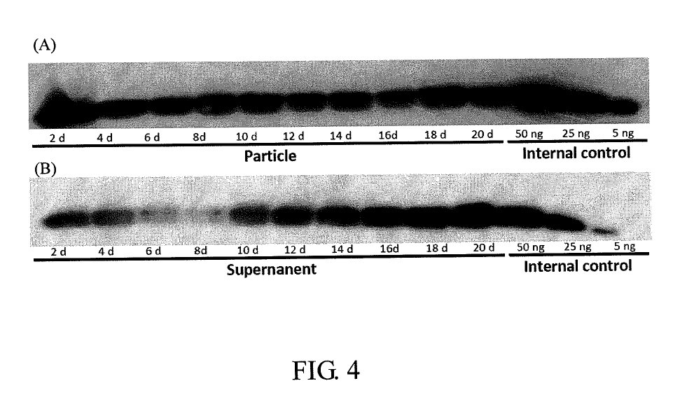

The amount of the released aFGF in one vial was monitored every another days during 20 days. The particles of the invention were contained in PBS at room temperature and the supernatant and pallet were collected after centrifugation for western blot test. The results showed that aFGF in the particles of the invention was slowly released into PBS for at least 20 days (see FIG. 4A and FIG. 4B). During the first eight days, the amount of aFGF decreased slowly but increased at the tenth day until the 20th day. The unreleased aFGF in the particles is still abundant on the 20th day according to the result from western blot. The decrease of aFGF during the first 8 days may be the result of the burst release at the beginning of the experiment and the constant degradation of aFGF. And the degradation of the particle itself may be the cause of accelerated aFGF release

### Example 4

### PC-12 in 6-OHDA Neurotoxin with aFGF in the Chitosan-Heparin Particles

### PC-12 Cell Culture

The neurotoxicity was test by PC-12 cells which were rat pheochromocytoma cell line. The cells were supplied by the Bioresource Collection and Research Center, Food Industry Research and Development Institute, Hsinchu, Taiwan, and maintained in RPMI 1640 supplemented with 10% HBS, 5% FBS, 1% penicillin/streptomycin (Gibco) at 37° C. in a humidified atmosphere containing 5% CO2 and the culture medium was changed every 2 days.

Neurotoxicity

6-hydroxydopamin (6-OHDA) is a neurotoxin, which leads to apoptosis of catecholaminergic cells. This pharmacological mechanism can be used to test neuron protecting efficiency of aimed drugs. The cells were seeded in 96 well plates at a density of 4×10⁴ cells/well, which were pre-coated with collagen. Following 24 hrs of starvation, the medium was changed into 5% serum. The experimental groups were given different treatments and added with 6-OHDA (sigma) until reaching the concentration of 100 uM. The medium without 6-OHDA works as control. The relative number of viable cells was monitored by MTT assay.

Cell viability (MTT) Assay

MTT (3-(4,5-Dimethylthiazol-2-yl)-2,5-diphenyltetrazolium bromide) was a tetrazolium salt that can be reduced to purple-colored formazan by normal cell. The stock solution of MTT (5 mg/ml) was added into each well to make the medium of 0.5 mg/ml MTT and cells were incubated for 4 h. The supernatant was removed to obtain the MTT metabolic product, formazan, which was then dissolved in 120 ul DMSO (dimethylsulfoxide, sigma) and placed on a shaking table for 5 min until thoroughly dissolved. 100 ul of dissolved-formazan from each well was transferred into another plate to measure the absorbance with 560 nm at background 670 nm.

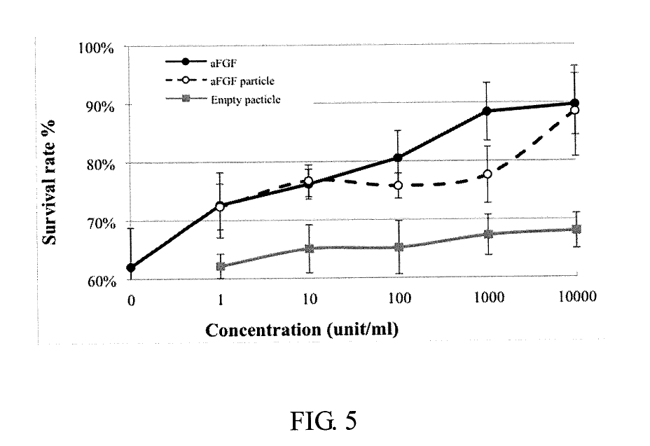

As shown in the results, the effect was dose-dependent, and the ED50 was about 125 uM. The dosage used was 100 uM, which caused about 40% apoptosis. 10 pg/ml aFGF increased the survival rate to more than 72.63%, and 88.41% with 10 ng/ml. As shown in FIG. 5, the result demonstrated that aFGF significantly blocks PC-12 death. The empty particles (without aFGF) and the particles entrapping an aFGF were soaked in culture medium for 2 days at 4° C. The supernatant was then collected and diluted at different concentrations for treatment of the 6-OHDA-damaged PC-12 cells. The apoptosis of the empty particle group with different dosage was all about 35% which was not significantly different from 6-OHDA treatment only. The aFGF-containing particles did not induce further cell death, which indicted that the chitosan/heparin particles were bio-safe for the application in tissue engineering. The aFGF released from the particles had good bioactivity to rescue the damaged-cell from apoptosis.

### Example 5

### Prevention of Abnormal Adhesion and Spinal Cord Injury Repair with aFGF

### Surgical Procedures and Animal Care

The adult female (250-300 g) Sprague-dawley rats were used in the study. All procedures involving animals were approved by the Animals Committee of the Taipei Veterans General Hospital. Animals were anesthetized with isoflurane before the lumbar spinal cord being exposed by laminectomy at the L1/L2 vertebral junction. The dorsal root entry zoon and the afferent nerve of L3-L6 spinal cord segments were revealed after piercing the dura matter with #5 Dummond forceps. The afferent nerve between the posterior root and dorsal root entry zoon was inflicted by forceps crush for three times, 10 sec each. Before closing the wound, the injury site was coated with particles either encapsulated with aFGF or not. The rat was kept at body temperature until it woke up.

Tissue Preparation

The animals were sacrificed by injecting over dose sodium pento-barbital intraperitoneally and perfused intracardially with 0.1 M phosphate buffer (PBS), following by 4% paraformaldehyde (PF) in 0.1 M PBS. The lumber spinal cord was removed and fixed in 4% PF overnight, and then cryoprotected with 15% sucrose for one day, followed by the overnight immersion of 30% sucrose at 4° C. Fixed specimens were embedded in OCT compound, snap-frozen and sectioned to 20 μm in thickness for staining and examination.

Immunohistochemistry

Immunohistochemistry technique was used to observe the regeneration of sensory axon by using calcitonin gene-related peptide (CGRP; 1:20,000; Sigma, St, Louis, Mo.) as the marker. The staining starts from 0.3% H202 infusion for 30 min to eliminate the endogeneses hydrogen peroxidase of the tissue. The samples were then incubated for 1 hour with 2% bovine serum albumin in PBS to block nonspecific binding. The slices were rinsed with PBS-Tween 20 (PBST) before primary antibody (anti-CGRP) incubation overnight at 4° C. The primary antibody labeled sections were washed in PBST three times, following by the protocol of Vectastain Elite ABC Kit (Vector Laboratories, Burlingame, Calif.) to attract secondary antibody and stained with DAB Substrate Kit (Vector Laboratories, Burlingame, Calif.). All images were captured using Olympus microscope with a cooling CCD system.

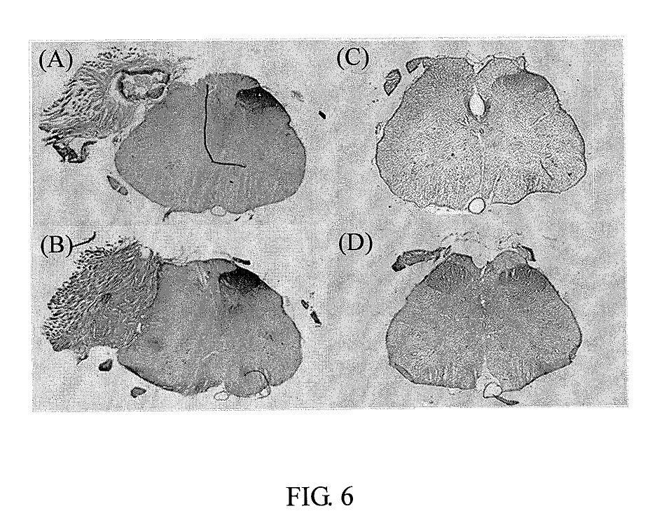

The spinal cord afferent pathway injury model was used to demonstrate the effect of particles in vivo. The rhizotomy was operated only on the left-side, and the right side remains intact as a control. Sensory axon fibers passed through DREZ to superficial lamina I-II of dorsal horn and was labeled by the anti-CGRP antibody (FIG. 6, the right side of spinal cord). Dorsal rhizotomy induced the degeneration of the sensory axon and hypertrophy of the tissue fibrosis (FIG. 6A and FIG. 6B). Following the injury, the sensory axon disappeared from the lamina of left dorsal horn and the scar tissue expanded around the spinal cord. The hypertrophic tissue invaded into the spinal cord and even the wound of the neural tissue in some cases. The aFGF-containing particles disclosed herein were able to reduce the fibrosis so as to keep the spinal cord structure intact (FIG. 6C and FIG. 6D). Rhizotomy reduced the mitogen-activated protein kinase to cause neural degeneration. It was indicated that aFGF was a potent cell mitogen for neural repair. In conclusion, the particles disclosed herein not only reduced the extensive fibrosis but also prevented the sensory axons from degeneration (FIG. 6D).

It will be appreciated by those skilled in the art that changes could be made to the embodiments described above without departing from the broad inventive concept thereof. It is understood, therefore, that this invention is not limited to the particular embodiments disclosed herein, but it is intended to cover modifications within the spirit and scope of the present invention as defined by the appended claims.

I/We claim:

1. A controlled release particle comprising a therapeutically effective amount of an acid fibroblast growth factor (aFGF) entrapped by a particle, which is composed of a biocompatible anionic biopolymer capable of binding to the aFGF and a cationic polymer.

2. The controlled release particle of claim 1, wherein the cationic polymer is chitosan.

3. The controlled release particle of claim 1, wherein the biocompatible anionic biopolymer is collagen, gelatin, alginate, heparin, or hyaluronan.

4. The controlled release particle of claim 1, where the biocompatible anionic biopolymer is heparin.

5. A method for manufacturing the controlled release particle of claim 1, comprising: mixing a biocompatible anionic biopolymer capable of binding to an aFGF and a cationic polymer to form a particle, and then with the aFGF to allow the aFGF to be entrapped by the particle.

6. The method of claim 5, wherein the cationic polymer is chitosan.

7. The method of claim 5, wherein the biocompatible anionic biopolymer is collagen, gelatin, alginate, heparin, or hyaluronan.

8. The method of claim 5, where the biocompatible anionic biopolymer is heparin.

9. A method for treating a nervous injury in a subject, comprising locally administrating to the subject at the nervous injury with the controlled release particle according to claim 1.

10. The method of claim 9, wherein the cationic polymer is chitosan.

11. The method of claim 9, wherein the biocompatible anionic biopolymer is collagen, gelatin, alginate, heparin, or hyaluronan.

12. The method of claim 9, where the biocompatible anionic biopolymer is heparin.

13. A controlled release particle, comprising a therapeutically effective amount of an acid fibroblast growth factor (aFGF) entrapped by a particle composed of heparin and chitosan.
